(19) United States
(12) Patent Application Publication
(10) Pub. No.: US 2012/0165325 A1
(43) Pub. Date: Jun. 28, 2012

(54) Stable forms of N-(2,6-Dimethyl-4-morpholin-4-yl-phenyl)-3,3-dimethyl-butyramide

(75) Inventors: Svend Treppendahl, Virum (DK); Karin Liltorp, Vanlose (DK); Heidi Lopez de Diego, Naerum (DK)

(73) Assignee: H. Lundbeck A/S, Valby-Copenhagen (DK)

(21) Appl. No.: 13/410,404

(22) Filed: Mar. 2, 2012

Related U.S. Application Data
(62) Division of application No. 12/777,935, filed on May 11, 2010.
(60) Provisional application No. 61/177,017, filed on May 11, 2009.

(30) Foreign Application Priority Data
Nov. 5, 2009 (DK) PA200900597

(51) Int. Cl. A61K 31/5375; A61P 25/24; A61P 25/18; C07D 295/135; A61P 25/08

(52) U.S. Cl. 514/231.2; 544/166

(57) ABSTRACT

Polymorphic forms of N-(2,6-dimethyl-4-morpholin-4-yl-phenyl)-3,3-dimethyl-butyramide are provided together with a process for the manufacture of said compound.

### FIELD OF THE INVENTION

The present invention relates to particular crystalline forms of N-(2,6-dimethyl-4-morpholin-4-yl-phenyl)-3,3-dimethyl-butyramide with improved properties.

### BACKGROUND OF THE INVENTION

The compound N-(2,6-Dimethyl-4-morpholin-4-yl-phenyl)-3,3-dimethyl-butyramide is disclosed in the international patent applications published as WO 2005/087754, WO 2007/090409 and WO 2009/015667. The compound is an opener of the KCNQ family potassium ion channels and as such useful in the treatment of diseases responsive to the opening of those channels.

Over 70 human genes encode potassium ion channels, and the KCNQ family is a particular family of potassium ion channels comprising five members (KCNQ1-5). Based on their widespread expression in the central nervous system (CNS), this family represents an interesting target for drug development. In fact, it has been shown that mutations in KCNQ2 and KCNQ3 channels seem to be responsible for an inherited form of epilepsy known as benign familial neonatal convulsion. It has also been reported that the compound retigabine, which is a KCNQ2 and KCNQ3 channel opener, successfully reduces the frequency of seizures in clinical trials (Neurol., 68, 1197-1204, 2007].

Oral dosage forms, and in particular tablets, are often preferred administration forms for the patient and the medical practitioner due to the case of administration and the resulting better compliance. For the preparation of tablets, it is generally preferred that the active ingredient is crystalline. A compound which exists in crystalline form may exist in more than one form, i.e. be polymorphic. Polymorphic forms may have distinct properties which significantly impact their applicability as drugs. In particular, polymorphic forms may exhibit different stability, solubility or dissolution rates, properties which need to be optimized in drug development.

The present invention relates to polymorphs of N-(2,6-dimethyl-4-morpholin-4-yl-phenyl)-3,3-dimethyl-butyramide with improved properties.

### SUMMARY OF THE INVENTION

The present inventors have found that specific polymorphs of the free base of N-(2,6-dimethyl-4-morpholin-4-yl-phenyl)-3,3-dimethyl-butyramide in crystalline form possess superior stability and/or dissolution rates.

Hence, in one embodiment, the invention relates to the free base of N-(2,6-dimethyl-4-morpholin-4-yl-phenyl)-3,3-dimethyl-butyramide in crystalline form with XRPD reflections at 10.36, 12.67, 28.64 and 29.89 (° 2θ).

In one embodiment, the invention relates to the free base of N-(2,6-dimethyl-4-morpholin-4-yl-phenyl)-3,3-dimethyl-butyramide in crystalline form with XRPD reflections at 8.68, 18.09, 22.60 and 30.62 (° 2θ).

In one embodiment, the invention relates to the free base of N-(2,6-dimethyl-4-morpholin-4-yl-phenyl)-3,3-dimethyl-butyramide in crystalline form with XRPD reflections at 8.63, 22.26, 23.40 and 30.49 (° 2θ).

In one embodiment, the invention relates to a pharmaceutical composition comprising the polymorphs of the present invention.

In one embodiment, the invention relates to a polymorph of the present invention for use as a medicament.

In one embodiment, the invention relates to a polymorph of the present invention for curing a disease selected from seizure disorders, schizophrenia, depressive disorders, and bipolar spectrum disorders.

In one embodiment, the invention relates to a polymorph of the present invention for use in therapy.

In one embodiment, the invention relates to a method for treating a disease which will benefit from opening of the KCNQ family potassium ion channels comprising the administration of a polymorph of the present invention to a patient in need thereof.

In one embodiment, the invention relates to a polymorph of the present invention for use in the treatment of diseases which will benefit from opening of the KCNQ family potassium ion channels.

In one embodiment, the invention relates to the use of a polymorph of the present invention in the manufacture of a medicament for the treatment of a disease which will benefit from opening of KCNQ family potassium ion channels.

In on embodiment, the invention relates to a process for the manufacture of N-(2,6-dimethyl-4-morpholin-4-yl-phenyl)-3,3-dimethyl-butyramide.

### FIGURES

FIG. 1: X-ray powder diffractogram of the α-form of N-(2,6-dimethyl-4-morpholin-4-yl-phenyl)-3,3-dimethyl-butyramide

FIG. 2: X-ray powder diffractogram of the β-form of N-(2,6-dimethyl-4-morpholin-4-yl-phenyl)-3,3-dimethyl-butyramide

FIG. 3: X-ray powder diffractogram of the γ-form of N-(2,6-dimethyl-4-morpholin-4-yl-phenyl)-3,3-dimethyl-butyramide

### DETAILED DESCRIPTION OF THE INVENTION

The present invention relates to polymorphic forms of the free base of N-(2,6-dimethyl-4-morpholin-4-yl-phenyl)-3,3-dimethyl-butyramide, the molecular structure of which is depicted below.

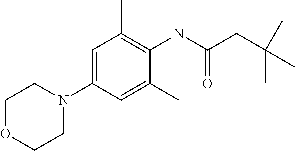

Each of these polymorphic forms is referred to as a polymorph of the present invention.

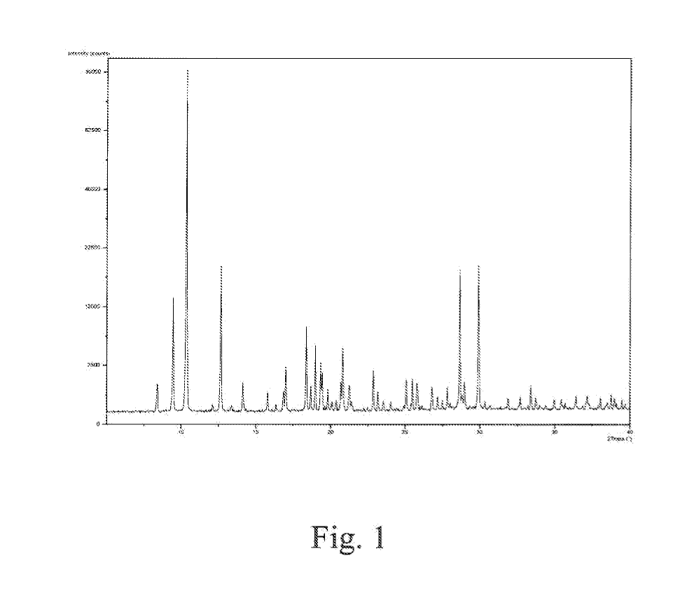

In one embodiment, the invention relates to crystalline N-(2,6-dimethyl-4-morpholin-4-yl-phenyl)-3,3-dimethyl-butyramide in a polymorphic form denoted herein as the α form, exhibiting X-ray powder diffraction (XRPD) reflections at 10.36, 12.67, 28.64 and 29.89 (2° θ). An XRDP of the α form is shown in FIG. 1. As shown in the examples, the α form has the lowest solubility (of the polymorphs of the present invention) from which observation it may be concluded that it is the most stable form.

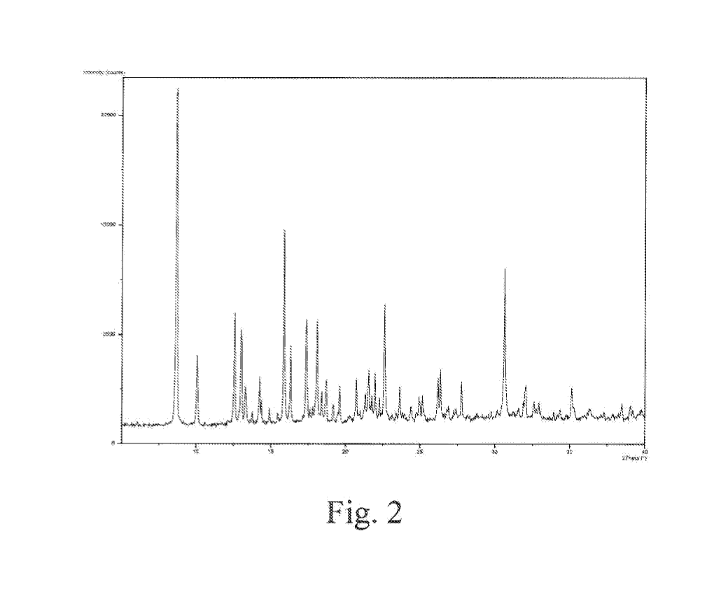

In one embodiment, the invention relates to crystalline N-(2,6-dimethyl-4-morpholin-4-yl-phenyl)-3,3-dimethyl-butyramide in a polymorphic form denoted herein as the β form exhibiting XRPD reflections at 8.68, 18.09, 22.60 and 30.62 (2° θ). An XRDP of the β form is shown in FIG. 2. As shown in the examples, the β form has a higher intrinsic dissolution rate than the α form. The β form is thus expected to enter into solution in the gastrointestinal tract more quickly than the α form and thus potentially give rise to a faster onset of action.

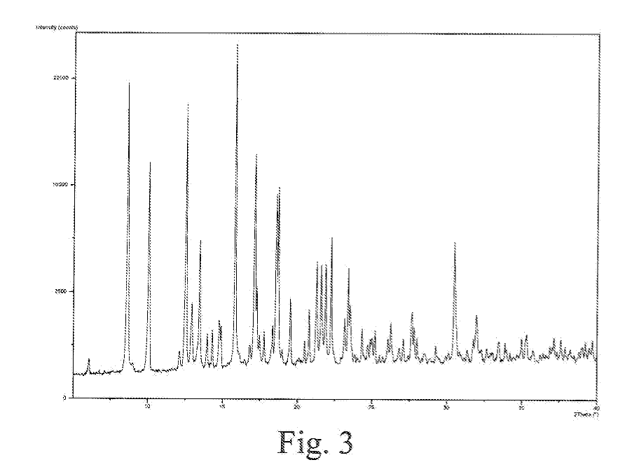

In one embodiment, the invention relates to crystalline N-(2,6-dimethyl-4-morpholin-4-yl-phenyl)-3,3-dimethyl-butyramide in a polymorphic form denoted herein as the γ form exhibiting XRPD reflections at 8.63, 22.26, 23.40 and 30.49 (2° θ). An XRDP of the γ form is shown in FIG. 3. As shown in the examples, the γ form is characterised by a faster intrinsic dissolution rate and is thus expected to enter into solution in the gastrointestinal tract more quickly than the α form and β-form and thus potentially give rise to a faster onset of action.

N-(2,6-dimethyl-4-morpholin-4-yl-phenyl)-3,3-dimethyl-butyramide may be synthesized as disclosed in WO 2005/087754, and the polymorphs of the present invention may be prepared as disclosed in the examples.

Alternatively, N-(2,6-dimethyl-4-morpholin-4-yl-phenyl)-3,3-dimethyl-butyramide may be manufactured by a process wherein a 4-halogen-2,6-dimethyl-aniline, such as 4-bromo-2,6-dimethyl-aniline is reacted in a solvent with 3,3-dimethyl-butyryl chloride preferably in the presence of a base, such as Na₂CO₃ to obtain N-(4-bromo-2,6-dimethyl-phenyl)-3,3-dimethyl-butyramide, or the corresponding 4-halogen compound.

Thus, in one embodiment, the invention relates to a process for the manufacture of N-(2,6-dimethyl-4-morpholin-4-yl-phenyl)-3,3-dimethyl-butyramide comprising reacting 4-halogen-2,6-dimethyl-aniline with 3,3-dimethyl-butyryl chloride in the presence of a base.

In one embodiment, 1 equivalent of 4-bromo-2,6-dimethyl-aniline is mixed with 1-2 equivalent, such as 1.5 equivalent Na₂CO₃ in tetrahydrofuran (TI-IF) under stirring and nitrogen atmosphere. After 1-2 hours, 1-1.5 equivalents, such as 1.1 equivalent 3,3-dimethyl-butyryl chloride is added, and stirring is continued until a desired degree of conversion has been achieved. The compound obtained may be worked up by phase extractions and re-crystallisations. Subsequently, this compound (or the corresponding 4-halogen compound) is reacted with morpholine in the presence of a palladium catalyst and a base to obtain N-(2,6-dimethyl-4-morpholin-4-yl-phenyl)-3,3-dimethyl-butyramide.

Hence, in one embodiment, the invention relates to a process for the manufacture of N-(2,6-dimethyl-4-morpholin-4-yl-phenyl)-3,3-dimethyl-butyramide comprising reacting N-(4-halogen-2,6-dimethyl-phenyl)-3,3-dimethyl-butyramide (such as the 4-bromo compound) with morpholine in the presence of a palladium catalyst and a base.

The palladium catalyst consists of a palladium source and a phosphine ligand. Useful palladium sources include palladium in different oxidation states, such as e.g. 0 and 11. Examples of palladium sources which may be used in a process of the present invention are Pd₂(dba)₃, Pd(dba)₂ and Pd(OAc)₂. dba designates dibenzylideneacetone and OAc designates acetate. Particular mention may be made of Pd(dba)₂. The palladium source is typically employed in an amount of 0.1-10 mol-%, such as 0.1-1 mol-%. Throughout this application, mol-% is calculated with respect to the limiting reactant.

Numerous phosphine ligands are known, including both monedentate and bidentate. Useful phosphine ligands include 2-(2-dicyclohexylphosphanylphenyl)-N,N-dimethylaniline (DavePhos), racemic 2,2′-bis-diphenylphosphanyl-[1,1′]binaphtalenyl (rac-BINAP), 1,1′-bis(diphenylphosphino)ferrocene (DPPF), bis-(2-diphenylphosphinophenyl)ether (DPEphos), tri-t-butyl phosphine (Fu's salt), biphenyl-2-yl-di-t-butyl-phosphine, biphenyl-2-yl-dicyclohexyl-phosphine, (2′-dicyclohexylphosphanyl-biphenyl-2-yl)-dimethyl-amine, [2′-(di-t-butyl-phosphanyl)-biphenyl-2-yl]-dimethyl-amine, and dicyclohexyl-(2′,4′,6′-tri-propyl-biphenyl-2-yl)-phosphane. Moreover, carbene ligands, such as e.g. 1,3-bis-(2,6-di-isopropyl-phenyl)-3H-imidazol-1-ium; chloride may be used instead of phosphine ligands. In one embodiment, the phosphine ligand is DavePhos. The phosphine ligand is typically added in an amount of 0.1-10 mol-%, such as 0.1-1 mol-%.

Base is added to the reaction mixture to increase pH. In particular, bases selected from NaO(t-Bu), KO(t-Bu) and Cs₂CO₃ are useful. Organic bases, such as 1,8-diazabicyclo[5.4.0]undec-7-ene (DBU) and 1,4-diazabicyclo[2.2.2]octane (DABCO) may be applied as well. Particular mention may be made of NaO(t-Bu) and KO(t-Bu). Typically, the base is added in an amount around 1-5 equivalents, such as 1-3 equivalents.

In one embodiment, 0.1-0.5 mol-%, such as 0.25 mol-% Pd(dba)₂ and 0.1-1 mol-%, such as 0.5 mol-% DavePhos, 1 equivalent of N-(4-bromo-2,6-dimethyl-phenyl)-3,3-dimethyl-butyramide and 1-2 equivalent, such as 1.6 equivalent of Na(Ot-Bu) are mixed with a solvent, such as dimcthoxyethane (DME), after which morpholine is added and the reaction is allowed to proceed until a desired degree of conversion is achieved. The final product may be worked up using phase extractions and re-crystallisations, and the final polymorphic form obtained may depend on the solvents used. As shown in the examples, re-crystallisation from water will result in the α-form.

As discussed above, KCNQ potassium ion channel openers have been shown to be useful in the treatment of seizure disorders, for which reason the polymorphs of the present invention may be useful in the treatment of acute seizures, convulsions, status epilepticus, and epilepsy, such as epileptic syndrome and epileptic seizures.

As shown in the examples, a number of relevant pre-clinical models indicate that the polymorphs of the present invention may be useful in the treatment of psychotic and mood diseases or disorders.

Psychotic diseases include schizophrenia. The symptoms of schizophrenia fall into four broad categories: positive, negative, cognitive and affective, such as depressive symptoms. The positive symptoms are those which manifest themselves as an ‘excess’ of normal behaviour, such as one or more of hallucinations, delusions, thought disorders, distortions or exaggerations in language and communication, disorganized speech, disorganized behaviour and agitation. The negative symptoms are those where patients show a lack of normal behaviour, such as one or more of blunted affect, aphasia, asociality, anhedonia, avolition, emotional withdrawal, difficulty in abstract thinking, lack of spontaneity, stereotyped thinking, alogia and attentional impairment. The cognitive symptoms relate to the cognitive deficits in schizophrenia, such as one or more of lack of sustained attention, deficits in executive function and memory. Affective symptoms of schizophrenia may include depressive symptoms, such as depressed mood in general, anhedonic symptoms, sleep disturbances, psychomotor agitation or retardation, sexual dysfunction, weight loss, concentration difficulties, delusional ideas, loss of energy, feelings of worthlessness, recurrent thoughts of death or suicidal ideation. Depressive symptoms in schizophrenia appear to be associated with a generally poor treatment outcome and are relatively frequent with an estimated prevalence of 25-60% (Montgomery and van Zwieten-Boot, Eur Neuropsychopharmacol., 2007, 17, 70-77).

Schizophrenia may be subdivided on the basis of the clinical picture. The paranoid subtype of schizophrenia is characterized by the presence of prominent delusions or auditory hallucinations in the context of a relative preservation of cognitive functioning and affect, whereas disorganized speech and behaviour, flat or inappropriate affect are essential features of the disorganized subtype of schizophrenia. The essential feature of the catatonic subtype of schizophrenia is a marked psychomotor disturbance that may involve both motoric immobility as well as excessive motor activity. Finally, the residual subtype of schizophrenia is characterized by a lack of prominent positive symptoms.

Mood disorders include disorders wherein a disturbance in mood is the predominant feature. Thus, both depressive disorders, such as major depressive disorder, dysthymic disorder, depressive disorder not otherwise specified, minor depression and brief recurrent depression mood disorders as well as bipolar spectrum disorders like bipolar 1 disorder, bipolar II disorder and cyclothymic disorder are classified as mood disorders. Major depressive disorder is a chronic recurring disease with considerable morbidity in the general population. The hallmark of the disease is a depressed mood. The clinical picture may be further characterised by anhedonic symptoms, sleep disturbances, psychomotor agitation or retardation, sexual dysfunction, weight loss, concentration difficulties and delusional ideas. However, the most serious complication of a depressive episode is that of suicidal ideation, leading to suicide attempts (DSM IV, American Psychiatric Association, Washington D.C. 1994). Besides major depressive disorder, other disorders are characterized by depressed mood, such as dysthymic disorder, depressive disorder not otherwise specified, minor depression and recurrent brief depressive disorder (DSM IV, American Psychiatric Association, Washington D.C. 1994). Dysthymic disorder is differentiated from major depressive disorder on the basis of severity, chronicity and persistence. Dysthymic disorder is characterized by chronic, less severe depressive symptoms that have been present for many years. The “depressive disorder not otherwise specified” category includes disorders with depressive features, like minor depressive disorder and recurrent brief depressive disorder that do not meet the criteria for other depressive disorders like major depressive disorder or dysthymic disorder. The essential feature of minor depression is one or more periods of depressive symptoms that are identical in duration to those expressed in major depressive disorder but which involve fewer symptoms and less impairment. Recurrent brief depression is characterised by recurrent brief episodes of depressive symptoms that are identical in number and severity to those expressed in major depressive disorder but with shorter duration.

Bipolar spectrum disorders, previously referred to as manic-depressive illness, are mood disorders where depressive symptoms are combined with at least one manic, hypomanic or mixed episode. A manic episode is characterised by a distinct period of abnormally and persistently elevated, expansive or irritable mood. A mixed episode is characterized by a period lasting at least one week in which both the criteria for a manic and major depressive episode are met. In similarity to a manic episode, a hypomanic episode is characterized by a distinct period during which there is an abnormally and persistently elevated, expansive or irritable mood. However, in contrast to a manic episode, a hypomanic episode is not severe enough to cause marked impairment in social or occupational functioning or to require hospitalisation and there are no psychotic features. The symptoms of a bipolar depressive episode are not different from those characterizing a major depressive episode. This is also the reason why many bipolar patients are initially diagnosed as suffering from major depression. As mentioned, it is the occurrence of manic, mixed or hypomanic episodes that gives rise to a bipolar diagnosis, which is distinct from a major depression diagnosis.

Bipolar spectrum disorders may be subdivided into bipolar I disorder, bipolar II disorder, cyclothymic disorder and bipolar disorder not otherwise specified. Bipolar I disorder is characterized by the occurrence of one or more manic or mixed episodes and often individuals have also had one or more major depressive episodes. Bipolar II disorder is characterized by the occurrence of one or more major depressive episodes accompanied by at least one hypomanic episode. Due to the progressive nature of bipolar I and II disorder, the patients experience an increasing risk of recurrence of symptoms with every new episode, as well as a growing risk of increasing duration and severity of subsequent episodes, if untreated. For this reason, both bipolar I and bipolar II disorder patients may eventually be classified as rapid cycling patients, where the patient experiences at least four episodes per year. Cyclothymic disorder is a sub-group of bipolar spectrum disorders where the mood disturbances are characterized by chronic, fluctuating mood disturbances involving numerous periods of hypomania and periods of depressive symptoms. Bipolar disorder not otherwise specified refers to a category of disorders with bipolar features that do not meet the criteria for any specified bipolar disorder mentioned above. Bipolar spectrum disorders are life-threatening conditions since patients diagnosed with a bipolar disorder have an estimated suicide risk that is 15 times higher than in the general population (Harris and Barraclough, 1997, British Journal of Psychiatry, 170:205-228). At present, bipolar spectrum disorders are treated by maintaining the bipolar patients on mood-stabilisers (mainly lithium or antiepileptics) and adding antimanic agents (lithium or antipsychotics) or antidepressants (tricyclic antidepressants or selective serotonin re-uptake inhibitors) when the patients relapse into a manic or depressive episode, respectively (Liebermann and Goodwin, Curr. Psychiatry Rep. 2004, 6:459-65). Thus, there is a desire to develop novel therapeutic treatments for bipolar spectrum disorders in order to meet the need of effectively treating all three crucial elements in these disorders with only one therapeutic agent: such novel agents should preferably alleviate manic symptoms with a fast onset of action (antimanic activity), alleviate depression symptoms with a fast onset of action (antidepressant activity), prevent the recurrence of mania as well as depression symptoms (mood stabilising activity).

Hence, in one embodiment, the invention relates to a method for the treatment of a disease selected from seizure disorders, psychotic diseases such as schizophrenia, depressive disorders and bipolar spectrum disorders, the method comprising the administration of an effective amount of a polymorph of the present invention to a patient in need thereof.

In one embodiment, the patient to be treated for any of the above-mentioned disease has initially been diagnosed with the disease.

A “therapeutically effective amount” of a polymorph of the present invention refers to an amount sufficient to cure, alleviate or partially arrest the clinical manifestations of a given disease and its complications in a therapeutic intervention comprising the administration of said compound. An amount adequate to accomplish this is defined as “a therapeutically effective amount”. Effective amounts for each purpose will depend on the severity of the disease or injury as well as on the weight and general state of the subject. It will be understood that determination of an appropriate dosage may be achieved using routine experimentation, by constructing a matrix of values and testing different points in the matrix, all of which is within the ordinary skills of a trained physician.

In the present context, the terms “disease”, “disorder” and “illness” are used as synonyms.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition, as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspects of the invention. The patient to be treated is preferably a mammal, in particular a human being.

Typically, the treatment of the present invention will involve daily administration of a polymorph of the present invention. This may involve once daily administration, or administration twice a day or even more frequently.

In an embodiment, the invention relates to a method wherein said polymorph is administered in an amount of between about 1 mg/day and 250 mg/day, such as about 1 mg/day, such as about 2.5 mg/day, such as about 5 mg/day, such as about 10 mg/day, such as about 50 mg/day, such as about 100 mg/day or about 250 mg/day. In one embodiment, a polymorph of the present invention may be administered as the only therapeutically effective compound.

In another embodiment, a polymorph of the present invention may be administered as a part of a combination therapy, i.e. the polymorph of the present invention may be administered in combination with one or more other therapeutically effective compounds having e.g. anti-convulsive properties or mood stabilising activity.

The present invention also relates to a pharmaceutical composition. A polymorph of the invention may be administered alone or in combination with pharmaceutically acceptable carriers or diluents, in either single or multiple doses. A pharmaceutical composition according to the invention may be formulated with pharmaceutically acceptable carriers or diluents as well as any other known adjuvants or excipients in accordance with conventional techniques, such as those disclosed in Remington: The Science and Practice of Pharmacy, 19 Edition, Gennaro, Ed., Mack Publishing Co., Easton, Pa., 1995.

A pharmaceutical composition of the invention may be specifically formulated for administration by any suitable route, such as the oral, rectal, nasal, pulmonary, topical, buccal, sublingual, transdermal, intracisternal, intraperitoneal, vaginal or parenteral (including subcutaneous, intramuscular, intrathecal, intravenous and intradermal) route, the oral route being preferred. It will be appreciated that the preferred route will depend on the general condition and age of the subject to be treated, on the nature of the disorder or disease to be treated, and on the active ingredient chosen. However, polymorphs of the present invention are particularly suited for the preparation of a solid dosage form, such as tablets.

Pharmaceutical compositions formed by combining a polymorph of the invention and the pharmaceutical acceptable carriers is then readily administered in a variety of dosage forms suitable for the disclosed routes of administration. The composition may conveniently be presented in unit dosage form employing methods known in the art of pharmacy.

Pharmaceutical compositions for oral administration may be solid or liquid. Solid dosage forms for oral administration include e.g. capsules, tablets, dragees, pills, lozenges, powders, and granules e.g. placed in a hard gelatine capsule in powder or pellet form or e.g. in the form of a troche or lozenge. Where appropriate, pharmaceutical compositions for oral administration may be prepared with coatings such as enteric coatings, or they can be formulated so as to provide controlled release of the active ingredient, such as sustained or prolonged release, according to methods well known in the art. Liquid dosage forms for oral administration include e.g. solutions, emulsions, suspensions, syrups and elixirs.

Compositions of the present invention suitable for oral administration may be presented as discrete units such as capsules or tablets, each containing a predetermined amount of the active ingredient, and which may include a suitable excipient. Furthermore, an orally available formulation may be in the form of powder or granules, a solution or suspension in an aqueous or non-aqueous liquid, or an oil-in-water or water-in-oil liquid emulsion.

Suitable pharmaceutical carriers include inert solid diluents or fillers, sterile aqueous solution and various organic solvent's. Examples of solid carriers are lactose, terra alba, sucrose, cyclodextrin, talc, gelatine, agar, pectin, acacia, magnesium stearate, stearic acid, lower alkyl ethers of cellulose, corn starch, potato starch, gums and the like. Examples of liquid carriers are syrup, peanut oil, olive oil, phospholipids, fatty acids, fatty acid amines, polyoxyethylene and water.

The carrier or diluent may include any sustained-release material known in the art, such as glyceryl monostearate or glyceryl distearate, alone or mixed with a wax.

Any adjuvants or additives usually used for such purposes, such as colouring agents, flavours, preservatives etc. may be used provided that they are compatible with the active ingredients.

The amount of solid carrier may vary but will usually be from about 25 mg to about 1 g.

Tablets may be prepared by mixing the active ingredient with ordinary adjuvants or diluents and subsequently compressing the mixture in a conventional tabletting machine.

The formulations may conveniently be presented in unit dosage form by methods known to those skilled in the art. A typical unit dosage form for oral administration one or more times per day, such as 1 to 3 times per day may contain from 0.01 to about 1000 mg, such as about 0.01 to 100 mg, preferably from about 0.05 to about 500 mg, and more preferably from about 0.5 mg to about 200 mg.

For parenteral routes, such as intravenous, intrathecal, intramuscular and similar routes of administration, typical doses are in the order of about half the dose employed for oral administration.

In one embodiment, the invention relates to a polymorph of the present invention for use in the treatment of a disease selected from seizure disorders, schizophrenia, depressive disorders and bipolar spectrum disorders.

In one embodiment, the invention relates to the use of a polymorph of the present invention for the manufacture of a medicament for the treatment of a disease selected from seizure disorders, schizophrenia, depressive disorders and bipolar spectrum disorders.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by con text).

### EXAMPLES

The melting points were measured by Differential Scanning calorimetry (DSC), using a TA-Instruments DSC-Q1000 instrument calibrated at 5°/min to give the melting point as onset value. About 2 mg of sample was heated 5°/min in a loosely closed pan under nitrogen flow.

X-Ray powder diffractograms (XRPD) were measured on a PANalytical X'Pert PRO X-Ray Diffractometer using CuK_{α1} radiation. The samples were measured in reflection mode in the 2θ-range 5-40° using an X'celerator detector. All values±0.1°.

### Example 1

### N-(2,6-Dimethyl-4-morpholin-4-yl-phenyl)-3,3-dimethyl-butyramide, α form

### Synthesis of N-(4-bromo-2,6-dimethyl-phenyl)-3,3-dimethyl-butyramide

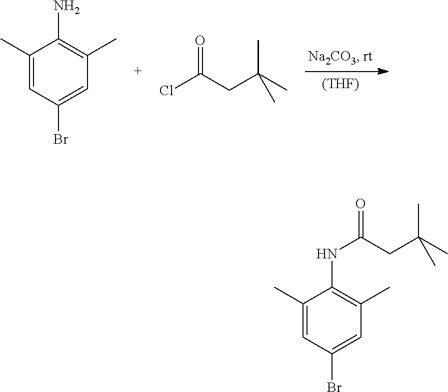

300 g (1.5 mol) 4-bromo-2,6-dimethylaniline and 239 g (2.25 mol, 1.5 eq) sodium carbonate in 1.25 L tetrahydrofuran were stirred under a nitrogen atmosphere at room temperature.

After 1 h, 230 mL (1.65 mol, 1.1 eq) 3,3-dimethylbutyryl chloride was added over a period of 2 h while the temperature was kept below 30° C., and the mixture was then stirred for 2 h at room temperature. 2.1 L tetrahydrofuran and 3.6 L water were added in order to get a clean phase separation. The water phase was extracted with 2.1 L tetrahydrofuran, and the combined organic phases were washed with 1.5 L 0.5 M aq. Na₂CO₃ solution. The solvent from the organic phase was distilled off and 2.1 L heptane was added to the resulting solid. The suspension was warmed to reflux, and allowed to cool down to room temperature. The solid was filtered off and washed with 300 mL heptane.

### Synthesis of N-(2,6-dimethyl-4-morpholin-4-yl-phenyl]-3,3-dimethyl-butyramide

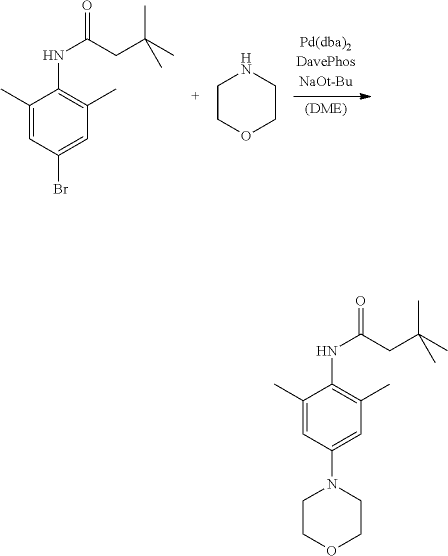

1.44 g (2.5 mmol, 0.0025 eq) bis-dibenzylideneacetone-palladium and 1.97 g (5.0 mmol, 0.005 eq) DavePhos, 298 g (1.0 mol) N-(4-bromo-2,6-dimethyl-phenyl)-3,3-dimethyl-butyramide and 154 g (1.6 mol, 1.6 eq) sodium-tert-butoxide were added to a nitrogen filled 3-neck 10 L round bottom flask. 2.0 L DME was added. 131 mL (1.5 mol, 1.5 eq) morpholine was added and the reaction mixture was warmed to reflux for 2-3 hours. 3.0 L water was added, and the resulting suspension was stirred overnight. The solid was filtered off and washed with 1.0 L water. The solid, together with 40 g charcoal, was then dissolved in 3 L 1 M aq. hydrochloric acid at reflux. After 1.5 h at reflux the reaction was blank filtered warm over filter aid and the filter was washed with 1.0 L warm water. The aqueous phase was washed with 1.0 L ethyl acetate and then with 1.0 L toluene. After phase separation, the aqueous phase was added to 690 g 27.7% aq. sodium hydroxide under vigorous stirring (pH 12-13), leading to precipitation. The resulting solid was filtered off and washed with 2.0 L water. The solid was dried in a vacuum oven at 40° C. for 72 h to give the title compound. (HPLC purity: 99.9% (area), XRPD diffractogram: α-polymorph).

### Example 2

### N-(2,6-Dimethyl-4-morpholin-4-yl-phenyl)-3,3-dimethyl-butyramide, β form

The β-form was obtained by heating the α-form to 170° C. with a heating rate of 10° C./min.

### Example 3

### N-[2,6-Dimethyl-4-morpholin-4-yl-phenyl]-3,3-dimethyl-butyramide, γ form

The γ-form was obtained by dissolving 1 g N-(2,6-Dimethyl-4-morpholin-4-yl-phenyl)-3,3-dimethyl-butyramide in 3 ml acetic acid at 70° C. Upon slow addition of 6 ml water (70° C.), the γ-form precipitates.

The tables below summarise properties of the α-, β- and γ-forms of N-(2,6-Dimethyl-4-morpholin-4-yl-phenyl)-3,3-dimethyl-butyramide free base.

| | | Solubility | Intrinsic |
|---|---|---|---|
| | | in water | dissolution |
| Polymorphic | Thermal properties/ | at RT at | rate (37C) |
| form | Tₒₙₛₑₜ (melting) | pH 7.4 | mg//cm²/min |
| α | Transforms into β. | 0.06 | 0.021 |
| | Endotherm on DSC | | |
| | around 150° C. when | | |
| | heated at 5° C./min reflects | | |
| | transformation into beta | | |
| β | 236.9° C. when covered. | 0.13 | 0.026 |
| | When uncovered it | | |
| | sublimates at around | | |
| | 200° C. | | |
| γ | 237.3° C. when covered. | | |
| | Whcn uncovered it | 0.13 | 0.036 |
| | sublimates at around | | |
| | 200° C. A small endotherm | | |
| | is seen around 145° C. prior | | |
| | to melting | | |

| Polymorphic form | Stability |
|---|---|
| α | Solid form stable for >90 days at 60° C. |
| | at a relative humidity of 95% |
| β | Solid form stable for >90 days at 60° C. |
| | at a relative humidity of 95% |
| γ | Solid form stable for >14 days at 60° C. |
| | at a relative humidity of 95% |

### Example 4

### Electrophysiology, Rat

Reports have suggested that inhibition of the number of spontaneously active dopaminergic neurones in the ventral tegmental area (VTA), i.e. the mesolimbic system, in rats may account for an antipsychotic potential of a compound (Chiodo and Bunney 1983, J. Neurosci., 5, 2539-2544.). In the mesolimbic system, all clinically used neuroleptics initially increase the firing rate of dopaminergic neurons (Ring et al., 1991, J. Neural Transm. Gen Sect., 84(1-2), 53-64,). After chronic administration, such neuroleptics eventually (after 3-4 weeks of treatment) decrease the firing rate to below pre-treatment levels (Skarsfeldt 1992, Synapse, 10, 25-33; White and Wang 1983, Science, 221, 1054-1057). This inhibitory effect on dopaminergic neurons, which is believed to be mediated by a depolarisation blockade, is thought to be of therapeutic significance to the antipsychotic effect of neuroleptics (Grace and Bunney 1986, J. Pharmacol. Exp. Ther. 238, 1092-1100). By inference, a compound that causes an acute decrease in spontaneous firing rate of mesolimbic dopaminergic neurones could be anticipated to possess a fast-onset antipsychotic potential. The presence of KCNQ subunits on DA neurons in the VTA in rodents is well-documented but their functionality is unknown (Saganich et al. 2001, J. Neurosci. 21(13)4609-4624; Cooper et al. 2001, J. Neurosci., 21(24)9529-9540). Consequently, it was studied in vivo whether the KCNQ opener of the present invention could acutely inhibit spontaneous activity of DA neurons in the VTA.

Subjects. Male Wistar rats (Harlan, The Netherlands) weighing 270-340 g were used. The animals were housed under a 12-hr light/dark cycle under controlled conditions for regular in-door temperature (21±2° C.) and humidity (55±5%) with food and tap water available ad libitum.

Experimental procedure. The rats were anaesthetised with an intraperitoneal injection of chloral hydrate (400 mg/kg). A femoral vein catheter was then inserted for supplementary anaesthetic injections (100 mg/kg) and drug administration. Animals were then mounted in a stereotaxic frame, the skull was exposed, and a hole (0.5×0.5 cm) was drilled above the ventral tegmental area. Extracellular single-cell recordings were performed using electrodes pulled from glass capillaries and filled with 2% Pontamine Sky Blue in 2 M NaCl. The tip of the electrode was broken under microscopic control, yielding an impedance of 2.0-8.0 MΩ a 135 Hz. The electrode was then lowered into the brain, using a hydraulic microdrive, aimed at the following coordinates: 5.5-5.0 mm posterior to Bregma; 0.5-0.9 mm lateral to the midline. Extracellular action potentials were amplified, discriminated and monitored on an oscilloscope and an audiomonitor. Discriminated spikes were collected and analysed using Spike 2 software (Cambridge Electronic Design Ltd., Cambridge, UK) on a PC-based system connected to a CED 1401 interface unit (Cambridge Electronic Design Ltd.). Presumed dopaminergic neurons were typically found 7.0-8.5 mm beneath the brain surface and were characterised by (1) a slow and irregular firing pattern (0.5-10 Hz), and (2) triphasic action potentials with a predominant positive component, a negative component followed by a minor positive component, with an overall duration >2.5 ms (Bunney et al. 1973, J. Pharmacol. Exp. They., 185, 560-571.).

Administration of compounds. Once a stable basal firing rate was obtained, cumulated doses of N-(2,6-Dimethyl-4-morpholin-4-yl-phenyl)-3,3-dimethyl-butyramide (dose range 0.03-0.5 mg/kg; volume range 0.12-1.0 ml/kg) were administered i.v., each injection being separated by at least 3 min. These i.v. doses match the s.c. dose range of 0-10 mg/kg.

Statistical analysis. Drug effects were assessed by statistical comparison of the mean firing rate calculated from the 2-3 min period immediately before the first drug administration (baseline) to the mean firing rate calculated from at least 60 s at the maximal drug effect. Data were analysed statistically by a one-way ANOVA followed by Student-Newman-Keuls posthoc test. A p-value less than 0.05 was considered significant.

Results. As can be seen from Table I, N-(2,6-dimethyl-4-morpholin-4-yl-phenyl)-3,3-dimethyl-butyramide significantly and dose-dependently inhibited the spontaneous DA cell firing in the VTA of anaesthetised rats following acute administration of compound. This data support the notion that this compound has a fast-onset antipsychotic potential.

TABLE 1
Effects on spontaneous DA cell firing in
the VTA of anaesthetised rats.

| | N-(2,6-Dimethyl-4- |
|---|---|
| Cumulated | morpholin-4-yl- |
| dose | phenyl)-3,3-dimethyl- |
| (mg/kg) | butyramide |
| 0 (Vehicle) | 97.5 ± 0.8 (10) |
| 0.03 | 89.8 ± 4.5 (5) |
| 0.1 | 81.0 ± 4.0 (5) ** |
| 0.25 | 74.1 ± 6.3 (4) *** |
| 0.3 | — |
| 0.5 | 68.1 ± 6.0 (4) *** |
| 0.6 | — |
| 0.9 | — |
| 1.0 | 57.1 ± 7.9 (3) *** |
| 2.0 | — |
| 4.0 | — |
| 6.0 | — |

Mean ± standard error of the mean.
Spontaneous DA cell firing rates expressed as a percentage of baseline firing rate;
n is indicated in brackets;
* p < 0.05,
** p < 0.01,
*** p < 0.001 compared to baseline (pre-drug administration activity).

### Example 5

### Amphetamine Challenge, Rat

D-amphetamine administration to rodents stimulates an increase in locomotor activity via mesolimbic dopamine receptors in the nucleus accumbens. While psychostimulant psychosis may not model all forms of schizophrenia, it may have applicability to paranoid schizophrenia and non-schizophrenic psychotic disorders (Krystal et al. pp. 214-224 in Neurobiology of Menial Illness, ISBN 0-19-511265-2). It is believed that inhibition of the amphetamine-induced increase in locomotor activity is a reliable method for the evaluation of compounds with an antipsychotic potential (Ogren et al., European J. Pharmacol. 1984, 102, 459-464). In the following experiment, it was tested if the inhibition of spontaneous DA neurons in the mesolimbic circuit that was assessed above could be translated into behavioral antipsychotic endpoint.

Subjects. Male Wistar rats (Taconic, Denmark) weighing 170-240 g are used. The animals were housed under a 12-hr light/dark cycle under controlled conditions for regular in-door temperature (21±2° C.) and humidity (55±5%) with food and tap water available ad libitum. Eight rats were used at each dose level and in the parallel control group receiving the vehicle to the test compound plus d-amphetamine and the group receiving vehicle injections only.

Experimental procedure. The experiment was made in normal light conditions in an undisturbed room. The test substance was injected 30 min before s.c. before the injection of d-amphetamine sulphate (0.5 mg/kg). Immediately after injection of d-amphetamine, the rats were placed individually in the test cages that were placed in a U-frame, equipped with 4 infrared light sources and photocells. The light beams crossed the cage 4 cm above the cage floor. Recording of a motility count required interruption of adjacent light beams, thus avoiding counts induced by stationary movements of the rat. Motility (counts) was recorded for a period of 2 hours. The mean motility induced by vehicle (saline) treatment in the absence of d-amphetamine was used as baseline. The 100 percent effect of d-amphetamine was accordingly calculated to be total motility counts minus baseline. The response in groups receiving test compound was thus determined by the total motility counts minus baseline, expressed in percent of the similar result recorded in the parallel amphetamine control group. The percent responses were converted to percent inhibition from which ED₅₀ values were calculated by means of log-probit analyses. In a parallel set of data, the potential sedative properties (motility inhibition) of the test compounds were evaluated using essentially the same procedure with the exception of not administering d-amphetamine-sulphate at the initiation of locomotor assessment.

Results. As can be seen from Table 2, N-(2,6-Dimethyl-4-morpholin-4-yl-phenyl)-3,3-dimethyl-butyramide produced an inhibition of the d-amphetamine induced hyperactivity in rats. The potency with which the effect was exerted was stronger than the potency to inhibit locomotor activity; that is, the inhibition of amphetamine-induced hyperactivity could not be explained by sedative properties of the compound. Rather, the efficacy reflects an antipsychotic potential N-(2,6-Dimethyl-4-morpholin-4-yl-phenyl)-3,3-dimethyl-butyramide. Since lithium is well accepted as efficacious for the treatment of acute mania and the prophylaxis of bipolar disorders (Goldberg 2000, J. Clin. Psychiatry 61 (Suppl. 13), 12-18), while olanzapine is accepted for its efficacy for the treatment of schizophrenia, and both lithium and olanzapine were efficacious in this model, these data support a potential N-(2,6-Dimethyl-4-morpholin-4-yl-phenyl)-3,3-dimethyl-butyramide to treat mania and bipolar disorder as well as schizophrenia.

TABLE 2
Effects of compounds on amphetamine-
induced hyperactivity in the rat.

| | Amphetamine | |
|---|---|---|
| | antagonism | Motility inhibition |
| | ED50 (mg/kg) ± | ED50 (mg/kg) ± |
| Compound | std. dev. | std. dev. |
| N-(2,6-Dimethyl-4- | 2.1 (1.5) | 7.6 (4.8) |
| morphohn-4-yl- | | |
| phenyl)-3,3-dimethyl- | | |
| butyramide | | |
| Lithium-chloride | 12 (1.7) | >40 |
| Olanzapine | 0.21 (1.7) | 0.72 (2.4) |

### Example 6

### Microdialysis, Rat

It is well-known that psychostimulants increase locomotor activity via an increase in extracellular DA levels in the nucleus accumbens, which is the terminal area of the mesolimbic DA projections (Guix et al., 1992, Neurosci. Lett., 138(1), 137-140; Moghaddam et al., 1989, Synapse, 4(2), 156-161). It is also known, that the antagonistic effect of antipsychotics on stimulant-induced hyperlocomotion is related to the effect of antipsychotics to inhibit the stimulated DA levels in the nucleus accumbens (Broderick et al., 2004, Prog. Neuropsychopharmacology and Biol. Psych., 28, 157-171). Thus, the nucleus accumbens is an accepted neuroanatomical site for testing reversal of positive symptoms of psychosis. Consequently, the following experiments were conducted to investigate the effect of N-(2,6-Dimethyl-4-morpholin-4-yl-phenyl)-3,3-dimethyl-butyramide on baseline and amphetamine-evoked levels of DA in the nucleus accumbens of freely moving rats. The experiments were conducted such that the data may be associated with the behavioural data obtained above.

Subjects. Male Sprague-Dawley rats (Charles River), initially weighing 275-300 g, were used. The animals were housed under a 12-hr light/dark cycle under controlled conditions for regular in-door temperature (21±2° C.) and humidity (55±5%) with food and tap water available ad libitum.

Surgery. Animals were anaesthetized with hypnorm/dormicum (2 ml/kg s.c.) and intracerebral guide cannulas (CMA/12) were stereotaxically implanted, positioning the dialysis probe tip in the nucleus accumbens (co-ordinates: 1.7 mm anterior to bregma, −1.2 mm lateral to bregma, 8.0 mm ventral to the dura). Anchor screws and acrylic cement was applied for fixation of the guide cannula. The body temperature of the animals was maintained at 37° C. by means of a rectal probe and a heating plate. The rats were allowed to recover from surgery for 2 days, housed singly in cages.

Experimental procedure. On the day of the experiment, a microdialysis probe (CMA/12, 0.5 mm diameter, 2 mm length) was inserted through the guide cannula of the conscious animal.

The probes were connected to a microinjection pump via a dual channel swivel which allowed the animals unrestricted movements. Perfusion of the microdialysis probe with filtered Ringer solution (145 mM NaCl, 3 mM KCl, 1 mM MgCl₂, 1.2 mM CaCl₂) was maintained for the duration of the experiment at a constant flow rate of 1 μL/min. After 180 min of stabilisation, the experiments were initiated. Dialysates were collected every 20 min. After the experiments the rats were sacrificed by decapitation, their brains removed, frozen and sliced for probe placement verification.

Administration of compounds. N-(2,6-dimethyl-4-morpholin-4-yl-phenyl)-3,3-dimethyl-butyramide (5 mg/kg) or vehicle (10% 2-hydroxy-propyl-beta-cyclodextrin, isotonic, pH 5-7) was administered subcutaneously in a volume of 2.5 ml/kg. Thirty min after the first administration dex-amphetamine sulphate (0.5 mg/kg s.c.) was administered.

Analysis of dialvsate. The concentration of dopamine (DA) in the dialysates was assessed by means of HPLC with electrochemicl detection. The dialysate constitutents were separated by reverse phase liquid chromatography (ODS 150×3 mm, 3 μM). Mobile phase consisted of 90 mM NaH₂PO₄, 50 mM sodium citrate, 367 mg/l sodium 1-octanesulfonic acid, 50 μM EDTA and 8% acetonitrile (pH 4.0) at a flow rate of 0.5 mL/min. Electrochemical detection of DA was accomplished using a coulometric detector; potential set at E1=−75 mV and E2=300 mV (guard cell at 350 mV) (Coulochem II, ESA). The dialysate levels of DA in the three dialyse samples preceding the administration of compound were averaged and used as baseline level of DA (100%).

Statistical analysis. The dialysate levels of DA in the three dialyse samples preceding the administration of compound were averaged and used as baseline level of DA (100%). Data were analysed using repeated measure analyses of variance followed by post hoc tests (Tukey test), when appropriate. *p<0.05 were considered significant.

Results. As can be seen in Table 3, N-(2,6-dimethyl-4-morpholin-4-yl-phenyl)-3,3-dimethyl-butyramide significantly (p=0.002) dampened the amphetamine-induced increase in extracellular levels of DA in the nucleus accumbens of freely moving rats. N-(2-6-dimethyl-4-morpholin-4-yl-phenyl)-3,3-dimethyl-butyramide did not significantly affected the basal extracellular DA level in this region (data not shown). These data suggest that the antagonistic effect of and N-(2,6-dimethyl-4-morpholin-4-yl-phenyl)-3,3-dimethyl-butyramide on amphetamine-induced activity in rats seen above, i.e. antipsychotic activity, is indeed associated with a dampening of provoked DA levels in the nucleus accumbens which further strengthens the antipsychotic potential of these compounds. The observation that merely provoked levels of DA were affected, but not basal levels of DA, suggests a low risk of causing anhedonia, a trait that is transiently, but frequently, observed with clinically used antipsychotics.

TABLE 3
Effects of compounds on the amphetamine-evoked
increase in DA levels in the nucleus accumbens
of freely moving rats.

| | | Amphetamine + N-(2,6- |
|---|---|---|
| | | dimethyl-4-morpholin-4-yl- |
| | | pheny1)-3,3-dimethyl- |
| | Amphetamine + | butyramide |
| Time | vehicle | (5 mg/kg) |
| (min) | % of baseline | % of baseline |
| −40 | 91 ± 6 | 108 ± 5 |
| −20 | 96 ± 5 | 100 ± 3 |
| 0 | 112 ± 7 | 91 ± 4 |
| 20 | 168 ± 19 | 112 ± 12 |
| 40 | 338 ± 27 | 227 ± 46 |
| 60 | 375 ± 46 | 262 ± 53* |
| 80 | 319 ± 59 | 195 ± 38* |
| 100 | 232 ± 48 | 172 ± 24 |
| 120 | 162 ± 37 | 166 ± 32 |
| 140 | 129 ± 27 | 129 ± 35 |

Normalised DA levels in the nucleus accumbens of freely moving rats are shown.
*P < 0.05 compared to amphetamine-vehicle group, same time.

### Example 7

### Amphetamine Sensitisation, Mouse

Clinical data imply that amphetamine-naïve schizophrenic and bipolar patients display an exaggerated response to a first dose of amphetamine implying that these patients may show a dopaminergic sensitisation (Strakowski et al. 1996, Biol. Psychiatry 40, 872-880, Lieberman et al. 1987, Psychopharmacology, 91, 415-433, Strakowski et al., 2001, CNS Drugs 15, 701-708). This phenomenon is modeled in rodents when repeated intermittent administration of amphetamine leads to a progressive increase in the behavioral response to an amphetamine challenge, a phenomenon known as behavioral sensitisation (Robinson and Berridge, Brain Research Rev. 1993, 18(3)247-91). The mesolimbic dopamine pathway is believed to be the major neural circuit involved in this behavioral sensitisation (Robinson and Becker, Brain Research 1986, 396(2):157-98). Inhibition of the behavioral response to an acute amphetamine challenge in sensitised animals is proposed as a model for evaluating the antipsychotic or antimanic potential of compounds.

Subjects. Male NMRI mice (Charles River) weighing approx. 35 g were used. The animals were housed 6 mice pr cage in a 12-hr light/dark cycle under controlled conditions for regular in-door temperature (21±2° C.) and humidity (55±5%) with food and tap water available ad libitum. 12 mice were used pr experimental group.

Experimental procedure. All mice were pre-treated once daily for five days with either d-amphetamine sulphate (2.5 mg/kg s.c.) or saline (10 ml/kg). For the 17 days between the last day of pre-treatment and the test day, the animals were kept in their home cage receiving the standard care as described above. The experiment was performed under normal light conditions in an undisturbed room. The mice were treated with test substance or vehicle and placed individually in the test cages for 30 min. The mice were then challenged with D-amphetamine sulphate (1.25 mg/kg s.c.) or saline (5 ml/kg) and replaced in the test-cage and data acquisition began. 5×8 infrared light sources and photocells interspaced by 4 cm monitor the locomotor activity. The light beams crossed the cage 1.8 cm above the bottom of the cage. The recording of a motility counted requires interruption of adjacent light beams, thereby avoiding counts induced by stationary movements of the mice.

Administration of compounds. Amphetamine-pretreated mice and vehicle-pretreated mice were s.c. treated with N-(2,6-dimethyl-4-morpholin-4-yl-phenyl)-3,3-dimethyl-butyramide (0-5 mg/kg) or vehicle (10% 2-hydroxy-propyl-beta-cyclodextrin, isotonic, pH 5-7, 5 ml/kg) 30 min prior to the data acquisition.

Data analyses. The total counts obtained in the 30 min test were averaged pr animal group and used for calculation of drug effects in the following manner: The average motility induced by an amphetamine challenge in amphetamine-pretreated animals was used as the sensitised response. The average motility induced by a vehicle challenge to vehicle-pretreated animals was used as a baseline motility response. The baseline value was subtracted from the sensitized amphetamine response value and set as 100% i.e. the sensitised response. This calculation was repeated for each dose group and the value for each dose-group is subsequently expressed relative to the 100% value. That is, the response in amphetamine-sensitized groups receiving test compound was thus determined as the sensitised response minus the baseline motility, expressed in percent of the similar result recorded in the sensitized amphetamine response group. The percent responses were converted to percent inhibition and exposed to log-probit analysis thus producing an ED₅₀ for inhibiting the sensitised response. Similarly, an ED₅₀ for inhibiting baseline motility was calculated by expressed the motility response in vehicle-pretreated, vehicle-challenged, drug-treated animals relative to the baseline motility response. A therapeutic index value can subsequently be calculated by dividing the first ED₅₀ by the second.

Results. As can be seen in Table 4, N-(2,6-dimethyl-4-morpholin-4-yl-phenyl)-3,3-dimethyl-butyramide as well as the antimanic compound lithium and the antipsychotic olanzapine all inhibit the hyperactivity induced by an amphetamine challenge in sensitised mice. The potency with which these compounds exert this effect is stronger than the potency with which these compounds inhibit baseline motility. That is, the compounds posses a calming effect, i.e. antipsychotic/antimanic effect, that is separable from its sedative effects (i.e. therapeutic index >1). This separation is characteristic for neuroleptics (Kapur and Mamo 2003, Biol. Psych. 27(7), 1081-1090) and thus support an antipsychotic/antimanic potential for N-(2,6-dimethyl-4-morpholin-4-yl-phenyl)-3,3-dimethyl-butyramide.

TABLE 4
Effects of compounds on a sensitised behavioural
response to amphetamine in mice.

| | Inhibition of | | |
|---|---|---|---|
| | amphetamine | Inhibition | |
| | sensitised | of baseline | |
| | response. | motility. | |
| | ED50 (±S.D.) | ED50 (±S.D.) | Therapeutic |
| Compound | (mg/kg) | (mg/kg) | index |
| N-(2,6-Dimethyl-4- | 1.6 (1.2) | >2.5 | >1 |
| morpholin-4-yl- | | | |
| phenyl)-3,3- | | | |
| dimethyl- | | | |
| butyramide | | | |
| Lithium-chloride | 34 (7.2) | >>40 | >>1 |
| Olanzapine | 0.11 (1.4) | >0.31 | >3 |

### Example 8

### Conditioned Avoidance, Rat

In the conditioned avoidance response (CAR) model, rats are trained to respond to a stimulus within a fixed time by moving from one place to another in order to avoid a footshock. Antipsychotics selectively suppress the avoidance response within a certain dose-range without suppressing escape behavior elicited by the appearance of the footshock. The CAR model is considered to be a predictive and reliable animal model that is sensitive to compounds with an antipsychotic potential. All clinically effective antipsychotics have been shown to inhibit CAR (Wadenberg and Hicks, Neuroscience and Biobehav Rev 23, 851-862, 1999).

Subjects. Male Wistar rats (Taconic, Denmark) weighing 150 g at the beginning of the study were used. The rats were housed in pairs and maintained on a 12 h light/dark cycle (lights on 06:00). The animals were fed once daily (approx. 6 pellets/rat) in order to keep the rats at 80% of their free-feeding weight. Water was available ad libitum. Temperature (21±1° C.) and relative humidity (55±5%) were automatically controlled.

Experimental procedure. Conditioned avoidance testing was conducted using four automated shuttle-boxes (ENV-010M, MED-Associates) each placed in a sound-attenuated chamber. Each box was divided into two compartments by a partition with an opening. The position of the animal and crossings from one compartment to the other were detected by two photocells placed on either side of the dividing wall. Upon presentation of the conditioned stimuli (CS), tone and light, the animals have 10 s to cross to the other compartment of the shuttle-box in order to turn the CS off (end the trial) and avoid the appearance of the unconditioned stimulus (UCS). If the rat remained in the same compartment for more than 10 s, the UCS is presented as 0.5 mA scrambled foot-shocks until escape was performed or 10 s in maximal duration. The following behavioural variables were evaluated: avoidance (response to CS within 10 s); escape (reponse to CS+UCS); escape failures (failure to respond); intertrial crosses and locomotor activity. The rats were habituated to the shuttle-box 3 min before each test session. During training each test session consists of 30 trials with intertrial intervals varying randomly between 20 s and 30 s. Training was carried until the rats display an avoidance of 80% or more, on 3 consecutive days. A test was preceded by a pre-test the day before giving rise to a baseline value for each animal, thus the animals served as their own control. Seven to eight rats were used at each dose level. A parallel control group receiving the vehicle of the test compound was also included.

Administration of compound. N-(2,6-dimethyl-4-morpholin-4-yl-phenyl)-3,3-dimethyl-butyramide (2.5 and 5 mg/kg) was administered s.c. 30 min before the test, in a volume of 5 ml/kg. The compound was dissolved in a vehicle of 10% 2-hydroxy-propyl-beta-cyclodextrin (isotonic with glucose, pH 5-7).

Statistical analyses. The effects of compounds on avoidance and escape failure behaviours were statistically evaluated by means of a two-way repeated measures ANOVA followed by post hoc comparisons (Student-Newman-Keuls Method) when appropriate. P-levels <0.05 were considered statistically significant.

Results. As can be seen in Table 5, N-(2,6-dimethyl-4-morpholin-4-yl-phenyl)-3,3-dimethyl-butyramide significantly reduced the number of avoidances. None of the tested doses caused any incidences of escape failures, corresponding to a lack of effect on motor performance (data not shown). In conclusion, these data support an antipsychotic potential of N-(2,6-dimethyl-4-morpholin-4-yl-phenyl)-3,3-dimethyl-butyramide.

TABLE 5
Effects of compounds on the conditioned
avoidance response in rats.

| | % inhibition of |
|---|---|
| | avoidance (Std. dev.) |
| Treatment | Relative to baseline. |
| Vehicle (10% Hpbeta) | −2 (4.2) |
| N-(2,6-dimethyl-4- | 1 (14) |
| morpholin-4-yl-phenyl)- | |
| 3,3-dimethyl- | |
| butyramide, 2.5 mg/kg | |
| N-(2,6-dimethyl-4- | 71 (26) *** P < 0.001 |
| morpholin-4-yl-phenyl)- | |
| 3,3-dimethyl- | |
| butyramide, 5 mg/kg | |

### Example 9

### Forced Swim Test, Mouse

The schizophrenic spectrum of symptoms involves a cluster of negative symptoms including anhedonia, social withdrawal and emotional flattening. Such symptoms are inadequately treated by currently available antipsychotics (Duncan et al. 2004, Schizoph. Res., 71(2-3), 239-248). The forced swim is test is a widely and frequently used model for preclinical evaluation of antipressant activity (Porsolt et al. 1977, Arch. Int. Phormacodyn. 229, 327-336). In order to test whether N-(2,6-dimethyl-4-morpholin-4-yl-phenyl)-3,3-dimethyl-butyramide has an antidepressant-like or mood elevating effect, the compound was tested in the mouse forced swim test.

Subjects. Male NMRI mice (Charles River) weighing 23-25 g were used. The mice were kept 8 mice pr cage in a 12-hr light/dark cycle under controlled conditions for regular in-door temperature (21±2° C.) and humidity (55±5%) with food and tap water available ad libitum. 8 mice were used pr experimental group.

Experimental procedure. The mice were placed in 2000 ml beaker containing 1200 ml of tempered water (25° C.) and left to swim for 6 min. The performance of the mice was video recorded, digitalized and analysed by means of a digital analysis system (Bioobserve). The time spent immobile for the last 3 min. of the test session was quantified for each mouse.

Treatment. 30 min. before the test, mice were treated s.c. with N-(2,6-dimethyl-4-morpholin-4-yl-phenyl)-3,3-dimethyl-butyramide or vehicle (10-%-2-OH-propyl-cyclodextrin, 10 ml/kg). In addition as positive control, imipramine-HCl (40 mg/kg) and a saline control (10 ml/kg) was included.

Analyses. The time spent immobile was statistically compared across the experimental groups against the relevant control group by means of one-way analysis of variance. A post-hoc test (Student-Newman-Keuls) was employed when appropriate. P-levels <0.05 were considered significant.

Results. As can be seen from Table 6, N-(2,6-dimethyl-4-morpholin-4-yl-phenyl)-3,3-dimethyl-butyramide significantly reduced the time spent immobile during the 3-6 min swim in mice. The efficacy was inferior to, yet comparable to, the effect of a relevant dose of imipramine-HCl. In contrast, the antipsychotic olanzapine had only a weak effect in this test which is in line with the observation that this compound has an inadequate effect on negative symptoms in humans. These data support an antidepressant potential of N-(2,6-dimethyl-4-morpholin-4-yl-phenyl)-3,3-dimethyl-butyramide which may translate into a potential to treat negative symptoms in schizophrenic patients.

TABLE 6
Effects of compounds on immobility in the
mouse forced swim test.

| | N-(2,6- | | | | | |
|---|---|---|---|---|---|---|
| | Dimethyl-4- | | | | | |
| | morpholin-4- | | | | | |
| | yl-phenyl)-3,3- | | | | | |
| | dimethyl- | | | | Imipramine- | |
| | butyramide | | Olanzapine | | HCl | |
| | Immobility in | | Immobility in | | Immobility in | |
| Dose: mg/kg | % (±S.D.) | | % (±S.D.) | | % (±S.D.) | |
| Vehicle | 100 | (6.6) | 100 | (6.61) | 100 | (7.1) |

| 0.31 | — | | 96 | (14) | — |
|---|---|---|---|---|---|
| 1.3 | 102 | (4.6) | 95 | (11) | — |

| | | | | |
|---|---|---|---|---|
| 2.5 | 96.7 | (7.5) | — | — |
| 5.0 | 82.3 | (20) * | — | — |

| | | | | |
|---|---|---|---|---|
| 40 | — | — | 73.8 | (22) * |

1-9. (canceled)

10. A method for treating a disease selected from seizure disorders, schizophrenia, depressive disorders, and bipolar spectrum disorders, the method comprising the administration of an effective amount of a compound N-(2,6-dimethyl-4-morpholin-4-yl-phenyl)-3,3-dimethyl-butyramide in a crystalline form with XRPD reflections at 10.36, 12.67, 28.64 and 29.98 (° 2θ) with XRPD reflections at 8.68, 18.09, 22.60 and 30.62 (° 2θ), or XRPD reflections at 8.63, 22.26, 23.40 and 30.49 (° 2θ) to a patient in need thereof.

11-13. (canceled)

14. A process for the manufacture of N-(2,6-dimethyl-4-morpholin-4-yl-phenyl)-3,3-dimethyl-butyramide comprising reacting 4-halogen-2,6-dimethyl-aniline with 3,3-dimethyl-butyryl chloride in the presence of a base.

15. The process according to claim 14, wherein said 4-halogen-2,6-dimethyl-aniline is 4-bromo-2,6-dimethyl-aniline.

16. A process for the manufacture of N-(2,6-dimethyl-4-morpholin-4-yl-phenyl)-3,3-dimethyl-butyramide comprising reacting N-(4-halogen-2,6-dimethyl-phenyl)-3,3-dimethyl-butyramide with morpholine in the presence of a palladium catalyst and a base.

17. The process according to claim 14, wherein said N-(4-halogen-2,6-dimethyl-phenyl)-3,3-dimethyl-butyramide is N-(4-bromo-2,6-dimethyl-phenyl)-3,3-dimethyl-butyramide.

18. The process according to claim 14, wherein 4-bromo-2,6-dimethyl-aniline is reacted with 3,3-dimethyl-butyryl chloride in the presence of a base to afford N-(4-bromo-2,6-dimethyl-phenyl)-3,3-dimethyl-butyramide, and wherein said N-(4-bromo-2,6-dimethyl-phenyl)-3,3-dimethyl-butyramide is subsequently reacted with morpholine in the presence of a palladium catalyst and a base to afford N-(2,6-dimethyl-4-morpholin-4-yl-phenyl)-3,3-dimethyl-butyramide.
